SEQUENCE LISTING (7 sequences)

SEQ ID NO: 1 - protein, 112 aa, M. bovis BCG
MAKKVTVTLVDDFDGSGAADETVEFGLDGVTYEIDLSTKNATKLRGDLKQWVAAGRRVGGRRRGRSGSGRGRGAIDREQSAAIREWARRNGHNVSTRGRIPADVIDAYHAAT

SEQ ID NO: 2 - DNA, 339 bp, M. bovis BCG
atggcgaagaaagtaaccgtcaccttggtcgacgatttcgacggttcgggcgccgccgacgaaacggtcgaattcgggcttgacggggtgacctatgagatcgacctttccactaagaatgccacgaaactgcgtggcgacctgaagcaatgggtggcggcgggccgtcgcgtcggtgggcgccgccgcggccgttccggatccggccgtggacgtggcgcgatcgaccgcgagcagagcgcggcgatccgcgaatgggctcgtcgtaacgggcacaatgtgtcgacgcgaggccggatcccggccgacgtcatcgacgcataccacgcggcgacctga

SEQ ID NO: 3 - DNA, 42 bp, Artificial Sequence
  primer
cggcttccataaattgggcagctggatcacctgctggcgcac

SEQ ID NO: 4 - DNA, 42 bp, Artificial Sequence
  primer
cggcttccatttcttggcatttggctaccggcgcccaggcga

SEQ ID NO: 5 - DNA, 44 bp, Artificial Sequence
  primer
cggcttccatagattggtggcttaccctcgcgtttcttcctgtg

SEQ ID NO: 6 - DNA, 44 bp, Artificial Sequence
  primer
cggcttccatcttttggggtgaagagatcacaccgcagacgacg

SEQ ID NO: 7 - DNA, 18 bp, Artificial Sequence
  primer
gccgtggccctacctggt

(19) United States
(12) Patent Application Publication
(10) Pub. No.: US 2015/0240201 A1
(43) Pub. Date: Aug. 27, 2015

(54) MODIFIED BCG STRAINS WITH REDUCED OR ELIMINATED ACTIVITY OF LSR2 AND PHARMACEUTICAL COMPOSITION COMPRISING SAME

(71) Applicants: Jun Liu, (US); Chengdu Yongan Pharmaceutical Co., Ltd., Chengdu (CN)

(72) Inventor: Jun Liu, Toronto (CA)

(73) Assignees: Chengdu Yongan Pharmaceutical Co., Ltd., Chengdu (CN); Jun Liu, Toronto, ON (CA)

(21) Appl. No.: 14/431,875

(22) PCT Filed: Sep. 27, 2012
(86) PCT No.: PCT/CN2012/082201

§ 371 Date: Mar. 27, 2015

(51) Int. Cl. C12N 1/20; A61K 39/04

(52) U.S. Cl. CPC C12N 1/20; A61K 39/04; A61K 2039/522

(57) ABSTRACT

The invention discloses a live modified Mycobacterium bovis-BCG strain in which the lsr2 gene is inactivated or its expression is reduced and a pharmaceutical composition comprising the same for the treatment or prophylaxis of a mammal against challenge by mycobacteria or against cancer. The invention further discloses a method for the treatment or prophylaxis of a mammal against challenge by Mycobacterium tuberculosis or Mycobacterium bovis or against cancer by administering to the mammal the live modified Mycobacterium bovis-BCG strain or the pharmaceutical composition of the present invention.

### FIELD OF THE INVENTION

This invention relates to tuberculosis (TB) vaccines. In particular, the invention provides a modified Bacille Calmette-Guérin (BCG) strain in which the lsr2 gene is inactivated or its expression is reduced.

### BACKGROUND OF THE INVENTION

Tuberculosis (TB), caused by Mycobacterium tuberculosis (M. tb), remains a global health threat. The latest surveillance data by the World Health Organization (WHO) reveals that in 2010, there were 8.8 million new cases and 1.4 million deaths from TB. Successful global TB control faces many obstacles including the difficulty of timely diagnosis, the lack of effective vaccines, and the fact that treatment requires many months of chemotherapy. The situation has been further complicated with the advent of M. tb/HIV coinfection and the emergence of multidrug-resistant (MDR) and extensively drug-resistant (XDR) TB. Because of these situations, effective approaches alternative to antibiotics are urgently needed for the control of TB. According to the Global Plan to Stop TB (2006-2015), the introduction of new, effective TB vaccines will be an essential component of any strategy to eliminate TB by 2050.

Bacille Calmette-Guérin (BCG), an attenuated strain of Mycobacterium bovis, is currently the only available vaccine for the prevention of TB. Since 1974, BCG vaccination has been included in the WHO Expanded Program on Immunization. More than 3 billion individuals have been immunized with BCG and >100 million doses of BCG are administered annually, making it the most widely used vaccine. Clinical studies have confirmed that BCG protects children, providing >80% efficacy against severe forms of TB, including meningitis and miliary TB (1, 2). However, BCG has a limited effect against pulmonary TB in adults with variable efficacy estimates from clinical studies ranging from 0 to 80% (3). Several hypotheses have been proposed to explain the variable efficacy, including differences in BCG strains used in clinical studies, differences in trial methods, differential exposure of trial populations to environmental mycobacteria, nutritional or genetic differences in human populations, and variations among clinical M. tb strains (4-9). These explanations are not mutually exclusive and all may contribute to the heterogeneity in BCG efficacy.

It is now clear that BCG is not an ideal vaccine and gives protection for only a limited period of time. The goal to develop a new and effective TB vaccine is to provide long-term protection. Existing BCG vaccines impart protection against the manifestations of TB in children, but their efficacy wanes over a period of 10 to 15 years, presumably because the protective immunity induced by BCG is gradually lost (10, 11). Currently, the consensus in the scientific filed is that the new generation of TB vaccines will work best using a heterologous prime-boost strategy to strengthen the immune response introduced by BCG (12, 13). This “prime-boost” strategy would include administration of a new recombinant BCG (rBCG), the “prime”, followed by a “booster” inoculation with a different vaccine (protein/peptide or DNA) to infants and young children before they are exposed to TB, or as a separate booster to young adults, or as an adjunct to chemotherapy (12, 13).

A key aspect of the issue concerns the immunogenicity of BCG vaccine. Numerous BCG strains are currently used as commercial vaccines (14). They are all descendants of the original M. bovis isolate that Calmette and Guérin passaged in vitro through 230 cycles during 1909-1921. Subsequent in vitro passages under different laboratory conditions around the world continued until 1960s, when the frozen seed lots were established (14). Because of the excessive in vitro passages (more than 1600 times for certain strains), it is thought that current BCG strains may have been over-attenuated thus not immunogenic enough to provide effective protection (15). The present invention describes a novel strategy to improve the efficacy of BCG.

### SUMMARY OF THE INVENTION

The immunogenicity of current BCG vaccine strains is not sufficient to induce the optimal protection in host against tuberculosis. However, based on our findings, a genetically engineered BCG strain in which lsr2 is inactivated or its expression reduced is more immunogenic and provides better protection.

Lsr2 is a small, basic protein highly conserved in mycobacteria including M. tb and M. bovis BCG (16). Previous studies by us and others showed that Lsr2 is involved in multiple cellular processes including cell wall lipid biosynthesis and antibiotic resistance (17, 18). Our biochemical studies demonstrated that Lsr2 is a DNA-binding protein and capable of bridging distant DNA segments (19). Moreover, we showed through in vivo complementation assays that Lsr2 is a functional analog of H-NS, a nucleoid associated protein of Enterobacteria (16).

More recently, our studies show that Lsr2 preferentially binds AT-rich sequences in mycobacterial genomes (20, 21). Our data revealed that Lsr2 negatively regulates the expression of 540 genes in M. tb genome, including many genes encoding important antigens (see Table 1). Because the genomes of M. bovis BCG, M. bovis and M. tb are >99.95% identical (22-24), these organisms are now called members of the Mycobacterium tuberculosis complex (MTBC) which refers to a genetically closely related group of Mycobacterium species that can cause tuberculosis. As such, deletion of lsr2 gene from a BCG strain or reducing lsr2 expression in a BCG strain will also lead to overexpression of multiple antigens. I hypothesize that such BCG strains will have enhanced immunogenicity and confer better protection against TB. This hypothesis is now confirmed by experimental evidence (see FIG. 1).

An exemplary amino acid sequence of Lsr2 is presented in SEQ ID NO: 1 in the sequence listing and an exemplary nucleotide sequence encoding the same is presented in SEQ ID NO: 2 in the sequence listing. These sequences represent Lsr2 from M. bovis BCG-Pasteur, as presented in the genome sequence available at the Pasteur Institute's BCGList Website (http://genolist.pasteur.fr/BCGList/).

Therefore, in one aspect, the present invention provides a modified Mycobacterium bovis BCG, in which lsr2 gene is inactivated by genetic engineering. In one embodiment, the lsr2 gene is inactivated by deleting the lsr2 gene from the genome. An example of constructing an lsr2 deletion mutant of BCG or M. tb is shown in FIG. 2.

In another aspect, the present invention also provides a modified Mycobacterium bovis BCG in which the expression of lsr2 is reduced. The modifications include but are not limited to: mutations of the promoter of lsr2 in the chromosomal DNA, expression of a dominant-negative Lsr2 mutant, expression of antisense lsr2 transcript, or expression of lsr2 knock-out constructs in an inducible promoter (e.g., tetracycline inducible promoter).

In one embodiment, the amino acid sequence of Lsr2 is shown in SEQ ID NO: 1 in the sequence listing and the nucleotide sequence encoding the same is shown in SEQ ID NO: 2 in the sequence listing.

In one embodiment, the Mycobacterium bovis-BCG strain is selected from the group consisting of Mycobacterium bovis-BCG-Russia, Mycobacterium bovis-BCG-Moreau, Mycobacterium bovis-BCG-Japan, Mycobacterium bovis-BCG-Sweden, Mycobacterium bovis-BCG-Birkhaug, Mycobacterium bovis-BCG-Prague, Mycobacterium bovis-BCG-Glaxo, Mycobacterium bovis-BCG-Denmark, Mycobacterium bovis-BCG-Tice, Mycobacterium bovis-BCG-Frappier, Mycobacterium bovis-BCG-Connaught, Mycobacterium bovis-BCG-Phipps, Mycobacterium bovis-BCG-Pasteur, and Mycobacterium bovis-BCG-China. All these BCG strains were derived from the same ancestor Mycobacterium bovis strain and are known to share similar properties (14). In addition, the mycobacteria of the invention need not be confined to strains of BCG. Those of skill in the art will recognize that other Mycobacterium strains may also be employed including attenuated strains of M. tb such as M. tb H37Ra.

In a further aspect, the invention provides a pharmaceutical composition for treatment or prophylaxis of a mammal against challenge by mycobacteria or against cancer comprising a modified Mycobacterium bovis-BCG strain in which lsr2 gene is inactivated. The pharmaceutical composition may further comprise a pharmaceutically acceptable carrier or an adjuvant or immunogenic materials from one or more other pathogens. In one embodiment, the pharmaceutical composition is a vaccine.

In another aspect, the invention provides a pharmaceutical composition for treatment or prophylaxis of a mammal against challenge by mycobacteria or against cancer comprising a modified Mycobacterium bovis-BCG strain in which the expression of lsr2 is reduced. The pharmaceutical composition may further comprise a pharmaceutically acceptable carrier or an adjuvant or immunogenic materials from one or more other pathogens. In one embodiment, the pharmaceutical composition is a vaccine.

Another aspect of this invention is to provide a method for the treatment or prophylaxis of a mammal against challenge by Mycobacterium tuberculosis or Mycobacterium bovis comprising: administering to the mammal a modified Mycobacterium bovis-BCG strain or a pharmaceutical composition of the instant invention. In one embodiment the mammal is a cow. In another embodiment the mammal is a human.

A further aspect of the invention is to provide a method for the treatment or prophylaxis of a mammal against cancer comprising: administering to the mammal a modified Mycobacterium bovis-BCG strain or a pharmaceutical composition of the current invention. In one embodiment the cancer is bladder cancer.

A still further aspect of the invention is to provide the use of the modified Mycobacterium bovis BCG in which lsr2 gene is inactivated or the expression of lsr2 is reduced of the invention in preparation of a medication for the treatment or prophylaxis of a mammal against challenge by mycobacteria or against cancer.

In one embodiment, the mycobacterium is Mycobacterium tuberculosis or Mycobacterium bovis.

### BRIEF DESCRIPTION OF THE DRAWINGS

FIG. 1. A graph shows that the lsr2 deletion mutant of BCG provides better protection than the parental BCG against virulent M. tb challenge.

FIG. 2. Schematic representation of major steps of constructing lsr2 deletion mutant of M. tb and BCG.

FIG. 3. Confirmation of lsr2 deletion mutants of M. tb and BCG generated using the method described above, wherein FIG. 3A shows the principle to confirm the lsr2 gene is successfully deleted from M. tb H37Rv and BCG-Japan; and FIG. 3B shows the electrophoresis result of the PCR products of the wild type M. tb H37Rv and BCG-Japan (lanes 1-2), and the lsr2 deletion mutants of M. tb H37Rv and BCG-Japan (lanes 3-8).

### DETAILED DESCRIPTION OF THE INVENTION

The present invention provides a vaccine or immune stimulating compositions, which includes one or more modified BCG strains. The modifications include: allelic inactivation of lsr2, expression of dominant-negative lsr2 mutant, or disruption of lsr2 promoter activity etc. These modifications will generate a modified BCG strain in which lsr2 is inactivated or its expression is reduced.

BCG is live, attenuated strain of M. bovis. It has long been known that administration of killed BCG strains results in a weak and transient immune response. However, it is recognized that the immunogenicity of current live BCG strains is also not optimal, which explains the failure of current BCG strains to provide effective protection. At present various strategies have been attempted to improve BCG immunogenicity, for example, by overexpressing antigen 85 (85A or 85B), or by expressing listerolysin in BCG to allow its escape into cytosol of infected macrophages for better antigen presentation (13). Both of these recombinant BCG strains have now entered clinical trials as new tuberculosis vaccine candidates (13).

However, M. tb contains more than 4,000 genes and many of which are immunogenic proteins (23). It is clear that the choices of antigens to be expressed in BCG to enhance its immunogenicity are far from complete and very often the choice of antigens for this purpose lacks a clear rationale. As such, researchers in the scientific community continue to search for new antigens or important genes for overexpression in BCG.

This invention is based on our present finding that deletion of lsr2 from M. tb leads to upregulation of numerous genes and many of which encode protective antigens (e. g., PE/PPE and ESX family proteins) (see Table 1), which offers a novel approach to augment the expression of multiple antigenic proteins. I suggest that by inactivating or reducing the activity of Lsr2 from a BCG strain, we are able to simultaneously increase the expression of multiple protective antigens, and such BCG will have enhanced immunogenicity and provide better protection against tuberculosis.

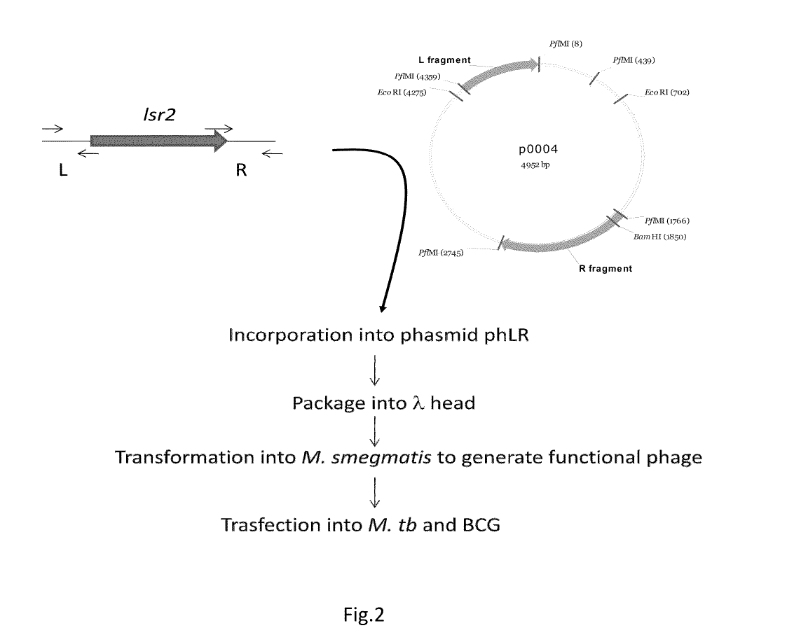

Despite recent studies of Lsr2, the effects of Lsr2 on gene expression in M. tb or BCG remain unknown due to the lack of lsr2 inactivated mutants in these organisms. The lsr2 gene in M. tb or BCG was thought to be essential and cannot be deleted; two other independent groups previously failed to obtain an lsr2 deletion mutant from M. tb or BCG, and consequently the authors concluded that lsr2 is essential in M. tb and BCG (18, 25). However, this was not formally proven (e.g., by introducing an extrachromosomal copy of lsr2 gene and demonstrating the successful deletion of the chromosomal lsr2). We have successfully obtained lsr2 deletion mutants of M. tb and BCG-Japan (see FIG. 2), which was generated by using a temperature-sensitive transducing phage system (26).

To determine the role of Lsr2 in gene regulation, we used the lsr2 deletion mutant of M. tb as an example and compared its transcriptional profile with the wild type M. tb strain. Microarray analysis shows that 540 genes are upregulated (2 fold) in the M. tb lsr2 deletion mutant compared to the wild type strain (see Table 1). A number of these genes encode potential antigens including 95 proteins associated with the cell wall and 22 PE/PPE family proteins which are known to be important antigens (Table 1) (23, 27). This result indicates that deletion of lsr2 increase the expression of multiple T cell antigens, which supports the key concept of my invention, that deleting lsr2 from a BCG strain increases the expression of multiple PE/PPE proteins and other protective antigens, providing an efficient means to enhance the immunogenicity and protective efficacy of BCG against tuberculosis.

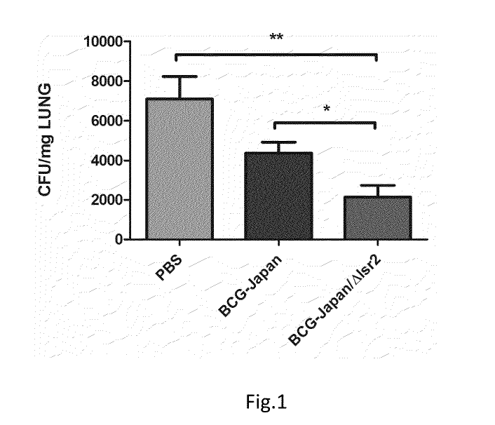

To confirm my hypothesis, we performed the animal infection experiments to assess the protective efficacy of the modified BCG. The result showed that the lsr2 deletion mutant strain of BCG confer significantly better protection than its parent BCG strain against M. tb challenge (FIG. 1). This result provides the proof of principle for the present invention. Because Lsr2 is highly conserved among mycobacteria (the sequence of Lsr2 is 100% identical between BCG and M. tb) (22, 23), deletion of lsr2 from other mycobacterial strains including attenuated strains of M. tb (e.g., M. tb H37Ra) is expected to generate a similar result and such strain may also be used as TB vaccine with improved protection efficacy.

M. bovis BCG is also used in the treatment of bladder cancer. Numerous randomized controlled clinical trials indicate that intravesical administration of BCG can prevent or delay tumor recurrence (28). The details of how BCG exerts this effect remain to be determined. However, the antitumor response requires an intact T-cell response, and involves increased expression of Th1-type cytokines, including TNF and IL-6 (29). As such, a BCG strain demonstrating increased immunogenicity may provide enhanced antitumor activity.

In summary, we use modified BCG strains with inactivated or reduced Lsr2 activity as vaccines to prevent TB and other mycobacterial infections. These modified BCG vaccines will induce better protective immunity against TB.

The modifications of lsr2 in a BCG strain may be carried out by any suitable method known in the art. Generally, the method of lsr2 inactivation will involve flanking an antibiotic resistance gene with nucleic acid sequences encoding parts of the Lsr2 protein and generate a knock-out construct. The replacement of the chromosomal copy of lsr2 gene will be achieved by allelic exchange. Those of skill in the art will recognize that many other methods are known and would be suitable for use in the invention. For example, the chromosomal lsr2 gene may be disrupted by transposon insertion or deletion from the chromosome. The methods of reducing the expression of Lsr2 include but are not limited to: overexpression of a dominant-negative Lsr2 mutant, expression of antisense Lsr2 transcript, and introducing mutations in the promoter regions of lsr2. In addition, overexpression of these genetic constructs may be inducible for example, under the tetracycline inducible promoters. Alternatively, genes that control the expression of lsr2 may also be targeted by genetic modifications to disrupt or reduce the Lsr2 activity.

Variations of Nucleic Acid Molecules

Modifications

Many modifications may be made to the nucleic acid molecule DNA sequences disclosed in this application and these will be apparent to one skilled in the art. The invention includes nucleotide modifications of the sequences disclosed in this application (or fragments thereof) that are capable of directing expression in bacterial or mammalian cells. Modifications include substitution, insertion or deletion of nucleotides or altering the relative positions or order of nucleotides.

Nucleic acid molecules may encode conservative amino acid changes in Lsr2. The invention includes functionally equivalent nucleic acid molecules that encode conservative amino acid changes and produce silent amino acid changes in Lsr2. Methods for identifying empirically conserved amino acid substitution groups are well known in the art (see for example, Wu, Thomas D. “Discovering Empirically Conserved Amino Acid Substitution Groups in Databases of Protein Families” (http://www.ncbi.nlm.nih.gov:80/entrez/query.fcgi?cmd=Retrieve&db=PubMed&list_uids=88775 23&dopt=Abstract).

Nucleic acid molecules may encode non-conservative amino acid substitutions, additions or deletions in Lsr2. The invention includes functionally equivalent nucleic acid molecules that make non-conservative amino acid changes within the amino acid sequences in Lsr2. Functionally equivalent nucleic acid molecules include DNA and RNA that encode peptides, peptides and proteins having non-conservative amino acid substitutions (preferably substitution of a chemically similar amino acid), additions, or deletions but which also retain the same or similar Lsr2. The DNA or RNA can encode fragments or variants of Lsr2.

Fragments are useful as immunogens and in immunogenic compositions.

Lsr2 like-activity of such fragments and variants is identified by assays as described below.

Sequence Identity

The nucleic acid molecules of the invention also include nucleic acid molecules (or a fragment thereof) having at least about: 60% identity, at least 70% identity, at least 80% identity, at least 90% identity, at least 95% identity, at least 96% identity, at least 97% identity, at least 98% identity or, most preferred, at least 99% or 99.5% identity to a nucleic acid molecule of the invention and which are capable of expression of nucleic acid molecules in bacterial or mammalian cells. Identity refers to the similarity of two nucleotide sequences that are aligned so that the highest order match is obtained. Identity is calculated according to methods known in the art. For example, if a nucleotide sequence (called “Sequence A”) has 90% identity to a portion of SEQ ID NO: 2, then Sequence A will be identical to the referenced portion of SEQ ID NO: 2 except that Sequence A may include up to 10 point mutations (such as substitutions with other nucleotides) per each 100 nucleotides of the referenced portion of SEQ ID NO: 2.

Sequence identity (each construct preferably without a coding nucleic acid molecule insert) is preferably set at least about: 70% identity, at least 80% identity, at least 90% identity, at least 95% identity, at least 96% identity, at least 97% identity, at least 98% identity or, most preferred, at least 99% or 99.5% identity to the sequences provided in SEQ ID NO: 2 or its complementary sequence). Sequence identity will preferably be calculated with the GCG program from Bioinformatics (University of Wisconsin). Other programs are also available to calculate sequence identity, such as the Clustal W program (preferably using default parameters; Thompson, J D et al., Nucleic Acid Res. 22:4673-4680), BLAST P, BLAST X algorithms, Mycobacterium avium BLASTN at The Institute for Genomic Research (http:tigrblast.tigr.org/), Mycobacterium bovis, M. Bovis BCG (Pastuer), M. marinum, M. leprae, M. tuberculosis BLASTN at the Wellcome Trust Sanger Institute (http://www.sarger.ac.uk/Projects/Microbes/), M. tuberculosis BLAST searches at Institute Pasterur (Tuberculist) (http://genolist.pasteur.fr/TubercuList/), M. leprae BLAST searches at Institute Pasteur (Leproma) (http://genolist.pasteur.fr/Leproma/), M. Paratuberculosis BLASTN at Microbial Genome Project, University of Minnesota (http://www.cbc.umn.edu/ResearchProjects/Ptb/and http://www.cbc.umn.edu/ResearchProjects/AGAC/Mptbhome.html), various BLAST searches at the National Center for Biotechnology Information—USA (http://www.ncbi.nlm.nih.gov/BLAST/) and various BLAST searches at GenomeNet (Bioinformatics Center—Institute for Chemical Research) (http://blast.genome.ad.jp/).

Since the genetic code is degenerate, the nucleic acid sequence in SEQ ID NO: 2 is not the only sequence which may code for a polypeptide having Lsr2 activity. This invention includes nucleic acid molecules that have the same essential genetic information as the nucleic acid molecules described in SEQ ID NO: 2. Nucleic acid molecules (including RNA) having one or more nucleic acid changes compared to the sequences described in this application and which result in production of the polypeptides shown in SEQ ID NO: 1 are within the scope of the invention. Other functional equivalent forms of Lsr2-encoding nucleic acids can be isolated using conventional DNA-DNA or DNA-RNA hybridization techniques.

Hybridization

The invention includes DNA that has a sequence with sufficient identity to a nucleic acid molecule described in this application to hybridize under stringent hybridization conditions (hybridization techniques are well known in the art). The present invention also includes nucleic acid molecules that hybridize to one or more of the sequences in SEQ ID NO: 2 or its complementary sequence. Such nucleic acid molecules preferably hybridize under high stringency conditions (see Sambrook et al. Molecular Cloning: A Laboratory Manual, Most Recent Edition, Cold Spring Harbor Laboratory Press, Cold Spring Harbor, N.Y.). High stringency washes have preferably low salt (preferably about 0.2% SSC) and a temperature of about 50-65° C.

Vaccines

One skilled in the art knows the preparation of live recombinant vaccines. Typically, such vaccines are prepared as injectable, either as liquid solutions or suspensions; solid forms suitable for solution in, or suspension in, liquid prior to injection may also be prepared. The preparation may also be emulsified, or the protein encapsulated in liposomes. The live immunogenic ingredients are often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants that enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn -glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80™ emulsion.

The effectiveness of an adjuvant may be determined by measuring the amount of antibodies directed against an immunogenic polypeptide containing a Mycobacterium tuberculosis antigenic sequence resulting from administration of the live recombinant Mycobacterium bovis-BCG vaccines that are also comprised of the various adjuvants. The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25%-70%.

The vaccines are administered in a manner compatible with the dosage formulation, and in such amount as will be prophylactically and/or therapeutically effective.

The vaccine may be given in a single dose schedule, or preferably in a multiple dose schedule. A multiple dose schedule is one in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. The dosage regimen will also, at least in part, be determined by the need of the individual and be dependent upon the judgment of the practitioner.

In addition, the live recombinant Mycobacterium bovis-BCG vaccine administered in conjunction with other immunoregulatory agents, for example, immune globulins. A subject of the present invention is also a multivalent vaccine formula comprising, as a mixture or to be mixed, a live recombinant Mycobacterium bovis-BCG vaccine as defined above with another vaccine, and in particular another recombinant live recombinant Mycobacterium bovis-BCG vaccine as defined above, these vaccines comprising different inserted sequences.

Pharmaceutical compositions

The pharmaceutical compositions of this invention are used for the treatment or prophylaxis of a mammal against challenge by Mycobacterium tuberculosis or Mycobacterium bovis. The pharmaceutical compositions of this invention are also used to treat patients having degenerative diseases, disorders or abnormal physical states such as cancer.

The pharmaceutical compositions can be administered to humans or animals by methods such as tablets, aerosol administration, intratracheal instillation and intravenous injection.

The present invention has been described in detail and with particular reference to the preferred embodiments; however, it will be understood by one having ordinary skill in the art that changes can be made without departing from the spirit and scope thereof. For example, where the application refers to proteins, it is clear that peptides and polypeptides may often be used. Likewise, where a gene is described in the application, it is clear that nucleic acids or gene fragments may often be used.

All publications (including Genbank entries), patents and patent applications are incorporated by reference in their entirety to the same extent as if each individual publication, patent or patent application was specifically and individually indicated to be incorporated by reference in its entirety.

### EXAMPLES

### Example 1

### Construction of lsr2 Deletion Mutant of M. tb and BCG

The lsr2 deletion mutants of M. tb H37Rv (a laboratory virulent strain of M. tb purchased from ATCC, ATCC no. 25618) and BCG-Japan (30) (a gift from Marcel Behr) were generated by using a temperature-sensitive transducing phage system (26) and the main steps are shown in FIG. 2. DNA manipulations were done essentially as described by Sambrook et al. (Sambrook, J., E. F. Fritsch, and T. Maniatis. 1989. Molecular cloning: a laboratory manual, 2nd ed. Cold Spring Harbor Laboratory, Cold Spring Harbor, NY.). Plasmid p0004 is a counterselectable suicide vector containing Hyg^{R}-sacB cassette (31). The upstream left fragment (L-fragment) and the downstream right fragment (R-fragment) flanking the lsr2 gene was generated by two primer pairs. The L-fragment (FIG. 2) for the allelic exchange substrate was generated by PCR using the primer pair L-forward

SEQ ID NO: 3
(CGGCTTCCATAAATTGGGCAGCTGGATCACCTGCTGGCGCAC)

### and L-reverse

SEQ ID NO: 4
(CGGCTTCCATTTCTTGGCATTTGGCTACCGGCGCCCAGGCGA).

The primer pair used for the R-fragment (FIG. 2) was R-forward

SEQ ID NO: 5
(CGGCTTCCATAGATTGGTGGCTTACCCTCGCGTTTCTTCCTGTG)

### and R-reverse

SEQ ID NO: 6
(CGGCTTCCATCTTTTGGGGTGAAGAGATCACACCGCAGACGACG).

The underlines indicate PfIMI restriction enzyme digestion sites. Since the genome regions flanking lsr2 in M. tb and BCG are identical, we used the M. tb genome DNA as template for the above PCR reaction to generate the knock out construct for both M. tb and BCG. The PCR reactions (50 μl) contain template DNA (10 ng), 0.5 μM primers, 0.2 mM dNTPs, 1× reaction buffer, 5% DMSO and 5 U Taq polymerase (Fermentas). The cycling conditions were: an initial 95° C. denaturation for 5 min, followed by 30 cycles of denaturation (95° C. for 30 sec), annealing (60° C., 30 sec), and extension (72° C., 1 min). A final extension at 72° C. for 5 min was used followed by cooling at 4° C. The resulting PCR products were run on agarose gel and purified using a gel purification kit (Qiagen). Purified L and R fragments and plasmid p0004 were digested with PfIMI (NEB) for 3 hour at 37° C. The digested L and R-fragments were gel purified using a gel purification kit (Qiagen). PfIMI cuts p0004 into 4 fragments and the two largest fragments (about 1600 and 1700 bp) were gel purified using the Qiagen gel purification kit. These two fragments were ligated with digested L and R-fragments obtained above to generate pKOlsr2 and transformed into E. coli DH5α. The ligation reaction (total 10 μl) contains 2 μl each of L and R-fragments, 2 μl each of the large fragments of p0004, 1 μl 10× T4 ligase buffer, 1 μl DNA T4 ligase (NEB). The ligation mixture was incubated at room temperature for 3 hours and then the reaction was inactivated by incubating at 65° C. for 20 min. The ligation mixture was added to competent E. coli DH5a cells and plated on LB agar containing hygromycin (150μg/ml). After overnight incubation at 37° C., single colonies were randomly picked and grown in LB broth. The plasmid pKOlsr2 was isolated from E. coli DH5a culture using a Qiagen Miniprep Kit. Purified pKOlsr2 was linearized by Pacl digestion and ligated to Pacl digested phasmid phLR (26). The ligation mixture contains 4 pKOlsr2, 4 μl phLR, 1 μl 10× T4 ligase buffer, 1 μl DNA T4 ligase (NEB). The ligation reaction proceeded at room temperature for 3 hours and then the resulting ligation product was packaged using the MaxPlax™ Lambda Packaging Extracts (Epicentre) and transformed into E. coli NM759 as the following. 5 μl of ligation mixture was added to 25 μl of the packaging extract and mix gently by tapping lightly with finger and incubated at room temperature for 2 hours. The reaction was stopped by adding 400 μl MP buffer (50 mM Tris HCl pH7.5, 150 mM NaCl, 10 mM MgSO₄, 2 mM CaCl₂) and incubated at room temperature for 10 min. Competent E. coli NM759 cells (1 mL) was then added to the mixture and incubated at 37° C. for 1 hour. The E. coli NM759 cells were pelleted and resuspended in 0.25 mL LB broth and 100 μl of which were plated on LB agar plates containing hygromycin (150 μg/ml) and incubated at 37° C. overnight. Single colonies were picked and grown in LB broth and the plasmid DNA was purified using a Qiagen Miniprep Kit. To generate and propagate functional phage, the phLR-pKOlsr2 purified from E. coli NM759 was transformed into Mycobacterium smegmatis (M. smegmatis) by electroporation. M. smegmatis (5 mL) were grown in Middlebrook 7H9 broth supplemented with 10% ADC (Difco) to OD₆₀₀ =0.8-1.0. M. smegmatis cells were washed three times with equal volume of 10% glycerol, each time by centrifugation and resuspension. After the final wash, the cells were resuspended in 0.5 mL 10% glycerol and immediately subjected to electroporation. To perform electroporation, 5 μl phLR-pKOlsr2 was added to 400μl of Mycobacterium smegmatis cultures in a BioRad 0.2cm cuvette, and electroporated at 2500V, 25 μFD, 10000Ω. These cells were then mixed with melted top agar and poured on Middlebrook 7H11 agar plates (Difco). After incubation at 30° C. for 4 days, 5 ml of MP buffer was then added to plates nearly confluent with plaques and rocked at room temperature for 4 hours to harvest functional phage. To perform phage transduction in M. tb or BCG, 20 ml M. tb or BCG culture grown in Middlebrook 7H9 broth supplemented with 10% ADC (Difco) was washed with buffer MP and then resuspended in 2 ml MP buffer. 0.5 ml phage obtained above was added to 1 ml of the M. tb or BCG cells and incubated overnight at 37° C. Subsequently the cells were spun and resuspended in 1 mL 7H9 broth containing 10% ADC (Difco) and incubated at 37° C. for 24 hours. Lastly the cells were spun down and plated on 7H11 agar containing 10% ADC and 50 μg/ml hygromycin and incubated at 37° C. for over 4 weeks.

### Example 2

### Confirmation of the Deletion of lsr2 Gene from M. tb H37Rv and BCG-Japan

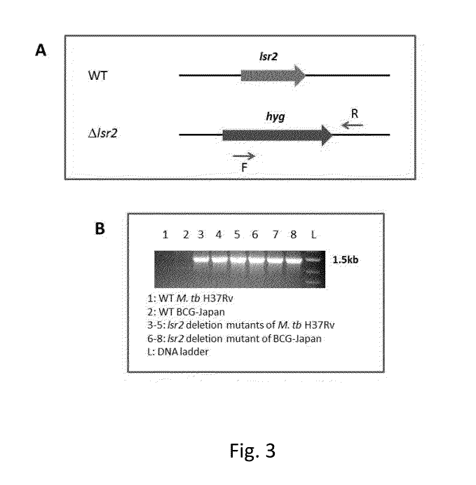

Three colonies of each strain (M. tb H37Rv and BCG-Japan) that appeared 4 weeks later from the above experiments were randomly picked and grown up in 20 mL 7H9 broth containing 10% ADC at 37° C. for 4 weeks. To isolate chromosomal DNA, 10 mL cultures of each were centrifuged at 2,000 ×g for 20 min, and the cell pellet was washed with 1 ml GTE Solution (25 mM Tris-HCl pH 8.0, 10 mM EDTA, 50 mM glucose) and resuspended in 450 μl GTE Solution. 50μl of lysozyme solution (10 mg/ml in Tris pH 8.5) was added, gently mixed, and incubated at 37° C. overnight. 100 μl 10% SDS and 50 μl 10 mg/ml Proteinase K (Sigma) were then added and gently mixed and incubated at 55° C. for 40 min. 200 μl 5 M NaCl and 160μl of CTAB were then added and gently mixed and incubated at 65° C. for 10 min. An equal volume (≈1 ml) chloroform:isoamyl alcohol (24:1) was added to the tube, the aqueous phase containing the DNA was transferred to a new tube and precipitated by adding 0.1 volume of 3 M sodium acetate, pH 5.2, and 1 volume of isopropanol. Invert the tube slowly to mix and place at 4° C. for 1 hour. Centrifuge the solution at 12,000×g for 30 min to pellet the DNA. Remove the supernatant and wash the DNA pellet with cold 70% ethanol. Centrifuge the DNA to remove the 70% ethanol and allow the pellet to air dry. Dissolve the pelleted chromosomal DNA in 100 μl TE buffer (10 mM Tris-HCl, pH 8.0, 1 mM EDTA). Chromosomal DNA of the wild type strains M. tb H37Rv and BCG-Japan were prepared by the same method and used as the control for PCR analysis. For PCR analysis, the primer pair forward (F) SEQ ID NO: 7 (GCCGTGGCCCTACCTGGT) and reverse (R) sequence SEQ ID NO: 6 (CGGCTTCCATCTTTTGGGGTGAAGAGATCACACCGCAGACGACG) were used. The forward primer was designed to detect the hyg cassette inserted in the chromosome of the lsr2 deletion mutant of M. tb H37Rv or BCG-Japan (see FIG. 3A) and the reverse primer was the same reverse primer used above to amplify the R fragment flanking the lsr2 gene. As such, an approximately 1.5 kb PCR product was expected from the lsr2 deletion mutant of M. tb H37Rv or BCG-Japan, which will not be generated from the wild type strain of M. tb H37Rv or BCG-Japan. The PCR reaction (50 μl) contains 0.5 μl of isolated chromosomal DNA as template, 5 μl each of the 10× forward and reverse primers, 1 μl Taq polymerase (Fermentas), 25 μl 2× PCR reaction buffer (Fermentas) and 13.5 μl dH₂O. The cycling conditions were: an initial 95° C. denaturation for 10 min, followed by 30 cycles of denaturation (95° C. for 1 min), annealing (58° C., 1 min), and extension (72° C., 1 min). A final extension at 72° C. for 5 min was used followed by cooling at 4° C. The resulting PCR products were run on agarose gel and detected by ethidium bromide staining (see FIG. 3B). Lanes 3-8 of FIG. 3B are randomly picked lsr2 deletion mutant colonies of M. tb or BCG generated by the above method and they all contained the expected ≈1.5 kb PCR products. By contrast, Lanes 1 and 2 are the wild type M. tb H37Rv and BCG-Japan, which did not produce the PCR product. This result confirmed that we have successfully obtained the lsr2 deletion mutants of M. tb H37Rv and BCG-Japan.

### Example 3

### Study of the Role of Lsr2 in Gene Regulation

Cultures (50 ml) of M. tb H37Rv wild type strain (WT) and M. tb Llsr2 (lsr2 deletion mutant obtained above) were grown in Middlebrook 7H9 broth supplemented with 10% ADC (Difco) and harvested at an OD₆₀₀≈0.4. Cells were pelleted and transferred to 2-ml screw cap tubes containing 1 ml RNA protect Bacterial Reagent (Qiagen) and incubated for 5 min at room temperature. Cells were again pelleted and resuspended in 400 μl lysis buffer (20 mM NaCH₃COOH, 0.5% SDS, 1 mM EDTA, pH 4) and 1 ml phenol/chloroform (pH 4.5, Sigma). Cells were disrupted by bead beating with glass beads by three 30-sec pulses using a bead beater (Biospec). They were then incubated at 65° C. for 4 min and then at 4° C. for 5 min before being centrifuged at 13,000 rpm for 5 min. The supernatant was then extracted with 300 μl of chloroform/isoamyl alcohol (24:1) and precipitated with isopropanol. Precipitated nucleic acids were collected by centrifugation and the pellets were washed with 70% ethanol and air dried. Crude RNA samples were treated with DNase I (Fermentas) for 2 hours at 37° C. and purified further using an RNeasy kit (Qiagen) according to the manufacturer's instructions. The quality of purified total RNA was assessed by gel electrophoresis. For cDNA production 25 μg total RNA was reverse transcribed at 42° C. overnight using 2 μl Superscript II reverse transcriptase (Invitrogen), 25 μg 9-mer random primers and 2 μl dNTP mix (0.5 mM dATP, 0.5 mM dCTP, 0.5 mM dGTP, 0.25 mM dTTP, 0.25 mM 5-(3-aminoalyl)-dUTP) in a total volume of 100 μl (25 mM Tris pH 8.4, 37.5 mM KCl, 3 mM MgCl₂, and 0.1 M DTT). RNA hydrolysis was performed by adding 15 μL 1M NaOH and then neutralized with 15 μL 1M HCl after incubating for 20 min at 65° C. The cDNA was purified using a QlAquick column (Qiagen). Samples were labeled for 1 hr at room temperature and then quenched with 4 M hydroxylamine. The labeled cDNA was purified and 1 μg per sample was hybridized to a 15 000 feature M. tb H37Rv ORF array with three distinct probes per ORF (Agilent Technologies) and scanned using the Genepix Professional 4200A scanner. Feature intensity ratios were acquired using Imagene v7.5 (Biodiscovery) and lowess-normalized using the marray R software package from Bioconductor. Significance Analysis of Microarrays (SAM) was performed to identify genes that are significantly upregulated or downregulated. The results were shown in table 1.

TABLE 1
List of 540 genes that are upregulated (≧2 fold) in the Isr2 deletion mutant of M. tb
H37Rv compared to the wild type strain.

| Rv # | Gene | Fold Change | Rv # | Gene | Fold Change |
|---|---|---|---|---|---|
| Rv1089 | PE10 | 41.27 | Rv1586c | Rv1586c | 8.14 |
| Rv2492 | Rv2492 | 39.03 | Rv0790c | Rv0790c | 8.14 |
| Rv1799 | lppT | 25.72 | Rv0966 | csoR | 7.80 |
| Rv1506c | Rv1506c | 24.94 | Rv2023A | Rv2023A | 7.80 |
| Rv0251c | hsp | 23.99 | Rv0448c | Rv0448c | 7.41 |
| Rv2107 | PE22 | 21.45 | Rv0862c | Rv0862c | 7.24 |
| Rv1115 | Rv1115 | 19.86 | Rv2643 | arsC | 7.19 |
| Rv1505c | Rv1505c | 17.60 | Rv0450c | mmpL4 | 7.13 |
| Rv3269 | Rv3269 | 17.06 | Rv2307B | Rv2307B | 6.99 |
| Rv0847 | lpqS | 16.03 | Rv2494 | Rv2494 | 6.97 |
| Rv2965c | kdtB | 15.66 | Rv1992c | ctpG | 6.72 |
| Rv3343c | PPE54 | 15.58 | Rv0678 | Rv0678 | 6.71 |
| Rv0791c | Rv0791c | 15.57 | Rv0767c | Rv0767c | 6.69 |
| Rv0976c | Rv0976c | 15.26 | Rv1502 | Rv1502 | 6.65 |
| Rv3888c | Rv3888c | 15.02 | Rv2913c | Rv2913c | 6.62 |
| Rv1089A | celA2a | 15.00 | Rv0232 | Rv0232 | 6.59 |
| Rv1587c | Rv1587c | 14.71 | Rv2516c | Rv2516c | 6.41 |
| Rv1504c | Rv1504c | 13.96 | Rv3887c | eccD2 | 6.34 |
| Rv1088 | PE9 | 12.95 | Rv0331 | Rv0331 | 6.34 |
| Rv3270 | ctpC | 12.88 | Rv0793 | Rv0793 | 6.28 |
| Rv1116 | Rv1116 | 11.85 | Rv3109c | moaA1 | 6.27 |
| Rv2035 | Rv2035 | 11.49 | Rv2034 | Rv2034 | 6.27 |
| Rv3054c | Rv3054c | 11.37 | Rv2338c | moeW | 6.14 |
| Rv1503c | Rv1503c | 11.02 | Rv0765c | Rv0765c | 6.04 |
| Rv1585c | Rv1585c | 10.90 | Rv0233 | nrdB | 6.02 |
| Rv0848 | cysK2 | 10.62 | Rv3841 | bfrB | 5.96 |
| Rv2108 | PPE36 | 10.61 | Rv2011c | Rv2011c | 5.94 |
| Rv0792c | Rv0792c | 10.18 | Rv3866 | espG1 | 5.93 |
| Rv0973c | accA2 | 10.04 | Rv3868 | eccA1 | 5.93 |
| Rv3864 | espE | 10.03 | Rv2745c | Rv2745c | 5.91 |
| Rv3161c | Rv3161c | 9.64 | Rv0677c | mmpS5 | 5.88 |
| Rv0507 | mmpL2 | 9.54 | Rv3406 | Rv3406 | 5.87 |
| Rv0405 | pks6 | 9.40 | Rv0475 | hbhA | 5.85 |
| Rv1522c | mmpL12 | 9.34 | Rv1576c | Rv1576c | 5.82 |
| Rv0449c | Rv0449c | 9.07 | Rv1800 | PPE28 | 5.80 |
| Rv1507c | Rv1507c | 8.95 | Rv3865 | espF | 5.77 |
| Rv0974c | accD2 | 8.62 | Rv0486 | mshA | 5.76 |
| Rv0508 | Rv0508 | 8.53 | Rv2012 | Rv2012 | 5.65 |
| Rv2466c | Rv2466c | 8.49 | Rv1255c | Rv1255c | 5.55 |
| Rv2339 | mmpL9 | 8.41 | Rv1356c | Rv1356c | 5.53 |
| Rv0451c | mmpS4 | 8.27 | Rv3867 | espH | 5.52 |
| Rv3160c | Rv3160c | 8.22 | Rv1471 | trxB1 | 5.44 |
| Rv2493 | Rv2493 | 5.43 | Rv0676c | mmpL5 | 4.35 |
| Rv2662 | Rv2662 | 5.43 | Rv2053c | Rv2053c | 4.34 |
| Rv0766c | cyp123 | 5.40 | Rv3060c | Rv3060c | 4.31 |
| Rv2780 | ald | 5.38 | Rv3020c | esxS | 4.27 |
| Rv2138 | lppL | 5.26 | Rv1507A | Rv1507A | 4.23 |
| Rv2517c | Rv2517c | 5.19 | Rv2659c | Rv2659c | 4.15 |
| Rv2658c | Rv2658c | 5.09 | Rv3902c | Rv3902c | 4.06 |
| Rv1582c | Rv1582c | 5.08 | Rv0837c | Rv0837c | 4.05 |
| Rv2036 | Rv2036 | 5.07 | Rv1405c | Rv1405c | 4.02 |
| Rv2642 | Rv2642 | 5.06 | Rv1894c | Rv1894c | 4.01 |
| Rv3251c | rubA | 5.01 | Rv0653 | Rv0654 | 4.00 |
| Rv2744c | 35kd_ag | 4.96 | Rv0982 | mprB | 3.99 |
| Rv3854c | ethA | 4.93 | Rv0244c | fadE5 | 3.92 |
| Rv3377c | Rv3377c | 4.90 | Rv2158c | murE | 3.92 |
| Rv0140 | Rv0140 | 4.90 | Rv3110 | moaB1 | 3.92 |
| Rv1581c | Rv1581c | 4.87 | Rv1057 | Rv1057 | 3.91 |
| Rv2664 | Rv2664 | 4.86 | Rv2016 | Rv2016 | 3.89 |
| Rv0586 | mce2R | 4.85 | Rv0324 | Rv0324 | 3.88 |
| Rv2337c | Rv2337c | 4.84 | Rv2827c | Rv2827c | 3.88 |
| Rv3886c | mycP2 | 4.80 | Rv2850c | Rv2850c | 3.85 |
| Rv0983 | pepD | 4.80 | Rv2023c | Rv2023c | 3.83 |
| Rv1584c | Rv1584c | 4.79 | Rv1501 | Rv1501 | 3.82 |
| Rv1221 | sigE | 4.78 | Rv0846c | Rv0846c | 3.82 |
| Rv1130 | prpD | 4.76 | Rv3288c | usfY | 3.82 |
| Rv3252c | alkB | 4.76 | Rv2727c | miaA | 3.81 |
| Rv2159c | Rv2159c | 4.67 | Rv3872 | PE35 | 3.78 |
| Rv0975c | fadE13 | 4.62 | Rv3085 | Rv3085 | 3.77 |
| Rv2491 | Rv2491 | 4.61 | Rv3884c | eccA2 | 3.77 |
| Rv3463 | Rv3463 | 4.59 | Rv1948c | Rv1948c | 3.77 |
| Rv3535c | hsaG | 4.58 | Rv3530c | Rv3530c | 3.76 |
| Rv3087 | Rv3087 | 4.57 | Rv3249c | Rv3249c | 3.76 |
| Rv2956 | Rv2956 | 4.55 | Rv0190 | Rv0190 | 3.75 |
| Rv0972c | fadE12 | 4.54 | Rv1222 | resA | 3.75 |
| Rv2963 | Rv2963 | 4.53 | Rv1895 | Rv1895 | 3.74 |
| Rv3574 | Rv3574 | 4.52 | Rv3534c | hsaF | 3.72 |
| Rv1955 | higA1 | 4.52 | Rv1490 | Rv1490 | 3.71 |
| Rv0764c | cyp51 | 4.49 | Rv1993c | Rv1993c | 3.71 |
| Rv3901c | Rv3901c | 4.48 | Rv0987 | Rv0987 | 3.69 |
| Rv1169c | PE11 | 4.46 | Rv1960c | parD1 | 3.69 |
| Rv2452c | Rv2452c | 4.43 | Rv0474 | Rv0474 | 3.69 |
| Rv0485 | Rv0485 | 4.39 | Rv2021c | Rv2021c | 3.67 |
| Rv3206c | moeB1 | 4.38 | Rv0970 | Rv0970 | 3.66 |
| Rv3286c | sigF | 4.36 | Rv3536c | Rv3536c | 3.65 |
| Rv3567c | hsaB | 3.65 | Rv2374c | hrcA | 3.19 |
| Rv0246 | Rv0246 | 3.65 | Rv3871 | eccCb1 | 3.17 |
| Rv2139 | pyrD | 3.65 | Rv1813c | Rv1813c | 3.15 |
| Rv3856c | Rv3856c | 3.64 | Rv1492 | mutA | 3.14 |
| Rv0769 | Rv0769 | 3.64 | Rv2866 | relE2 | 3.13 |
| Rv0563 | htpX | 3.64 | Rv2728c | Rv2728c | 3.12 |
| Rv3382c | lytB1 | 3.63 | Rv1801 | PPE29 | 3.11 |
| Rv1991c | mazF6 | 3.62 | MTB000043 | metU | 3.11 |
| Rv2829c | vapC22 | 3.62 | Rv2052c | Rv2052c | 3.11 |
| Rv1265 | Rv1265 | 3.60 | Rv3873 | PPE68 | 3.10 |
| Rv0411c | glnH | 3.59 | Rv1131 | gltA1 | 3.09 |
| Rv1218c | Rv1218c | 3.58 | Rv0971c | echA7 | 3.08 |
| Rv0988 | Rv0988 | 3.58 | Rv0402c | mmpF1 | 3.08 |
| Rv3849 | EspR | 3.56 | Rv3079c | Rv3079c | 3.08 |
| Rv3383c | idsB | 3.54 | Rv2323c | Rv2323c | 3.07 |
| Rv0384c | clpB | 3.52 | Rv2558 | Rv2558 | 3.07 |
| Rv0968 | Rv0969 | 3.52 | Rv0850 | Rv0850 | 3.07 |
| Rv0940c | Rv0940c | 3.51 | Rv0865 | mog | 3.06 |
| Rv2017 | Rv2017 | 3.50 | Rv2234 | ptpA | 3.05 |
| Rv1959c | parE1 | 3.45 | Rv0674 | Rv0674 | 3.04 |
| Rv0836c | Rv0836c | 3.45 | Rv1635c | Rv1635c | 3.04 |
| Rv2641 | cadI | 3.41 | Rv1969 | mce3D | 3.04 |
| Rv1989c | Rv1989c | 3.40 | MTB000021 | rrf | 3.03 |
| Rv0063 | Rv0064 | 3.39 | Rv0047c | Rv0047c | 3.02 |
| Rv1579c | Rv1579c | 3.39 | Rv2729c | Rv2729c | 3.02 |
| Rv2707 | Rv2707 | 3.38 | Rv3515c | fadD19 | 3.01 |
| Rv3857c | Rv3857c | 3.36 | Rv3289c | Rv3289c | 3.01 |
| Rv3061c | fadE22 | 3.36 | Rv1285 | cysD | 3.00 |
| Rv2324 | Rv2324 | 3.36 | Rv1224 | tatB | 2.98 |
| Rv3582c | ispD | 3.34 | Rv2743c | Rv2743c | 2.97 |
| Rv0671 | lpqP | 3.34 | Rv2499c | Rv2499c | 2.93 |
| Rv1854c | ndh | 3.33 | Rv0587 | yrbE2A | 2.93 |
| Rv1817 | Rv1817 | 3.32 | Rv2602 | Rv2602 | 2.93 |
| Rv1956 | higB1 | 3.31 | MTB000004 | glyU | 2.93 |
| Rv0763c | Rv0763c | 3.30 | Rv1583c | Rv1583c | 2.91 |
| Rv0672 | fadE8 | 3.29 | Rv1641 | infC | 2.90 |
| Rv3627c | Rv3627c | 3.29 | Rv2020c | Rv2020c | 2.89 |
| Rv3424c | Rv3424c | 3.29 | Rv0768 | aldA | 2.88 |
| Rv0789c | Rv0789c | 3.28 | Rv2912c | Rv2912c | 2.88 |
| Rv1463 | Rv1463 | 3.27 | MTB000009 | thrV | 2.87 |
| Rv3833 | Rv3833 | 3.23 | Rv3838c | pheA | 2.87 |
| Rv0849 | Rv0849 | 3.22 | Rv3862c | whiB6 | 2.86 |
| Rv3088 | Rv3088 | 3.19 | Rv1985c | Rv1985c | 2.86 |
| Rv3615c | espC | 2.86 | Rv1048c | Rv1048c | 2.61 |
| Rv0477 | Rv0477 | 2.85 | Rv1832 | gcvB | 2.60 |
| Rv2878c | mpt53 | 2.84 | Rv0991c | Rv0991c | 2.59 |
| Rv2710 | sigB | 2.83 | Rv0770 | Rv0770 | 2.58 |
| Rv2399c | cysT | 2.83 | Rv0320 | Rv0320 | 2.58 |
| Rv3616c | espA | 2.81 | Rv0947c | Rv0947c | 2.58 |
| Rv2133c | Rv2133c | 2.81 | Rv3570c | hsaA | 2.58 |
| Rv2656c | Rv2656c | 2.79 | Rv3188 | Rv3188 | 2.57 |
| Rv2160c | Rv2160c | 2.79 | Rv3869 | eccB1 | 2.57 |
| Rv3870 | eccCa1 | 2.78 | Rv0122 | Rv0123 | 2.56 |
| Rv2096c | Rv2096c | 2.77 | Rv3250c | rubB | 2.56 |
| Rv3086 | adhD | 2.77 | Rv2164c | Rv2164c | 2.56 |
| Rv1767 | Rv1767 | 2.76 | Rv3408 | Rv3408 | 2.55 |
| Rv0670 | end | 2.75 | Rv0024 | Rv0024 | 2.54 |
| Rv1603 | hisA | 2.75 | Rv2665 | Rv2665 | 2.54 |
| Rv1961 | Rv1961 | 2.74 | Rv0272c | Rv0272c | 2.54 |
| Rv0514 | Rv0514 | 2.74 | Rv1066 | Rv1066 | 2.54 |
| Rv0366c | Rv0366c | 2.73 | Rv2156c | murX | 2.53 |
| Rv0771 | Rv0771 | 2.73 | Rv3245c | mtrB | 2.53 |
| Rv0193c | Rv0193c | 2.72 | Rv0589 | mce2A | 2.53 |
| Rv0327c | cyp135A1 | 2.72 | Rv3855 | ethR | 2.53 |
| Rv0328 | Rv0328 | 2.71 | Rv2366c | Rv2366c | 2.52 |
| Rv3052c | nrdI | 2.71 | Rv3094c | Rv3094c | 2.52 |
| Rv0990c | Rv0990c | 2.71 | Rv1996 | Rv1996 | 2.51 |
| Rv1627c | Rv1627c | 2.69 | Rv1812c | Rv1812c | 2.51 |
| Rv0602c | tcrA | 2.69 | Rv0275c | Rv0275c | 2.51 |
| Rv2557 | Rv2557 | 2.69 | Rv2025c | Rv2025c | 2.50 |
| Rv1326c | glgB | 2.68 | Rv0922 | Rv0922 | 2.50 |
| Rv0412c | Rv0412c | 2.68 | Rv2022c | Rv2022c | 2.49 |
| Rv0874c | Rv0874c | 2.68 | Rv3287c | rsbW | 2.49 |
| Rv1219c | Rv1219c | 2.68 | MTB000031 | glyV | 2.49 |
| MTB000032 | argW | 2.67 | Rv1063c | Rv1063c | 2.49 |
| Rv3608c | folP1 | 2.67 | Rv2050 | Rv2050 | 2.48 |
| Rv1403c | Rv1403c | 2.66 | Rv2142c | parE2 | 2.47 |
| Rv0350 | dnaK | 2.66 | Rv1103c | mazE3 | 2.47 |
| Rv3066 | Rv3066 | 2.65 | Rv3084 | lipR | 2.46 |
| Rv1727 | Rv1727 | 2.65 | Rv0277c | Rv0277c | 2.46 |
| Rv3552 | Rv3552 | 2.65 | Rv1256c | cyp130 | 2.46 |
| Rv2504c | scoA | 2.65 | Rv3614c | espD | 2.46 |
| Rv2826c | Rv2826c | 2.64 | Rv0326 | Rv0326 | 2.45 |
| Rv2161c | Rv2161c | 2.64 | Rv168c | PPE17 | 2.45 |
| Rv0834c | PE_PGRS14 | 2.64 | MTB000017 | leuX | 2.45 |
| Rv3531c | Rv3531c | 2.62 | Rv2254c | Rv2254c | 2.44 |
| Rv3053c | nrdH | 2.44 | Rv0596c | Rv0596c | 2.32 |
| Rv3309c | upp | 2.44 | Rv0688 | Rv0688 | 2.31 |
| Rv1073 | Rv1073 | 2.44 | Rv0969 | ctpV | 2.31 |
| Rv3294c | Rv3294c | 2.44 | Rv1957 | Rv1957 | 2.31 |
| Rv0749 | Rv0749 | 2.43 | Rv3082c | virS | 2.30 |
| Rv0591 | mce2C | 2.43 | Rv0653c | Rv0653c | 2.30 |
| Rv3899c | Rv3899c | 2.42 | Rv3093c | Rv3093c | 2.30 |
| Rv2650c | Rv2650c | 2.42 | RvOO11c | Rv0011c | 2.30 |
| Rv1600 | hisC1 | 2.42 | MTB000026 | mpB | 2.30 |
| Rv1335 | Rv1335 | 2.42 | Rv1084 | Rv1084 | 2.29 |
| Rv3111 | moaC1 | 2.42 | Rv2631 | Rv2631 | 2.29 |
| Rv1665 | pks11 | 2.42 | Rv2663 | Rv2663 | 2.29 |
| Rv2552c | aroE | 2.42 | Rv2145c | wag31 | 2.29 |
| Rv0577 | TB27.3 | 2.41 | Rv2601A | Rv2601A | 2.28 |
| Rv0576 | Rv0576 | 2.40 | Rv0823c | Rv0823c | 2.27 |
| Rv1621c | cydD | 2.39 | Rv3529c | Rv3529c | 2.27 |
| Rv1533 | Rv1533 | 2.39 | Rv0302 | Rv0302 | 2.27 |
| Rv2957 | Rv2957 | 2.39 | Rv0134 | ephF | 2.27 |
| Rv2655c | Rv2655c | 2.39 | Rv1908c | katG | 2.27 |
| Rv3347c | PPE55 | 2.38 | Rv3613c | Rv3613c | 2.26 |
| Rv2918c | glnD | 2.38 | Rv3410c | guaB3 | 2.26 |
| Rv2243 | fabD | 2.37 | Rv2657c | Rv2657c | 2.26 |
| Rv0403c | mmpS1 | 2.37 | Rv0490 | senX3 | 2.26 |
| Rv3057c | Rv3057c | 2.36 | Rv1682 | Rv1682 | 2.25 |
| Rv0188 | Rv0188 | 2.36 | Rv1947 | Rv1947 | 2.25 |
| Rv3612c | Rv3612c | 2.36 | Rv2989 | Rv2989 | 2.25 |
| Rv0826 | Rv0826 | 2.36 | Rv3555c | Rv3555c | 2.24 |
| Rv1090 | celA2b | 2.36 | Rv3384c | Rv3384c | 2.24 |
| Rv1464 | csd | 2.35 | Rv2746c | pgsA3 | 2.24 |
| Rv3569c | bphD | 2.35 | MTB000006 | thrT | 2.24 |
| Rv2676c | Rv2676c | 2.35 | Rv1968 | mce3C | 2.24 |
| Rv3562 | fadE31 | 2.35 | Rv3350c | PPE56 | 2.23 |
| Rv1040c | PE8 | 2.35 | Rv3823c | mmpL8 | 2.22 |
| Rv3334 | Rv3334 | 2.34 | Rv3416 | whiB3 | 2.22 |
| Rv2706c | Rv2706c | 2.33 | MTB000039 | gluU | 2.22 |
| Rv2426c | Rv2426c | 2.33 | Rv1982c | Rv1982c | 2.21 |
| Rv3724B | cut5b | 2.33 | Rv2351c | plcA | 2.21 |
| Rv0459 | Rv0459 | 2.32 | Rv2731 | Rv2731 | 2.21 |
| Rv2134c | Rv2134c | 2.32 | Rv0864 | moaC2 | 2.21 |
| Rv0757 | phoP | 2.32 | Rv2459 | Rv2459 | 2.21 |
| Rv3426 | PPE58 | 2.32 | Rv1776c | Rv1776c | 2.21 |
| Rv2498c | citE | 2.32 | Rv2566 | Rv2566 | 2.21 |
| Rv0989c | grcC2 | 2.32 | Rv2553c | Rv2553c | 2.21 |
| Rv0488 | Rv0488 | 2.20 | Rv3549c | Rv3549c | 2.12 |
| Rv2651c | Rv2651c | 2.20 | Rv3540c | ltp2 | 2.12 |
| Rv3197A | whiB7 | 2.20 | Rv1990c | Rv1990c | 2.12 |
| Rv0804 | Rv0804 | 2.20 | Rv1967 | mce3B | 2.11 |
| Rv3537 | Rv3537 | 2.20 | Rv0465c | Rv0465c | 2.11 |
| Rv1936 | Rv1936 | 2.20 | Rv0986 | Rv0986 | 2.11 |
| Rv2769c | PE27 | 2.20 | Rv3327 | Rv3327 | 2.11 |
| Rv1577c | Rv1577c | 2.19 | Rv2483c | plsC | 2.11 |
| Rv1223 | htrA | 2.19 | Rv3724A | cut5a | 2.11 |
| Rv0647 | Rv0648 | 2.19 | Rv0113 | gmhA | 2.10 |
| Rv1540 | Rv1540 | 2.18 | Rv1986 | Rv1986 | 2.10 |
| Rv1580c | Rv1580c | 2.18 | Rv2779c | Rv2779c | 2.10 |
| Rv2801c | mazF9 | 2.18 | Rv1398c | Rv1398c | 2.10 |
| Rv0467 | icl | 2.18 | Rv1039c | PPE15 | 2.10 |
| Rv0500A | Rv0500A | 2.18 | Rv0070c | glyA2 | 2.08 |
| Rv1217c | Rv1217c | 2.17 | Rv1339 | Rv1339 | 2.08 |
| Rv0921 | Rv0921 | 2.17 | Rv0397 | Rv0397 | 2.08 |
| Rv1461 | Rv1461 | 2.17 | Rv3083 | Rv3083 | 2.08 |
| Rv3480c | Rv3480c | 2.16 | Rv1601 | hisB | 2.08 |
| Rv2687c | Rv2687c | 2.16 | Rv1619 | Rv1619 | 2.08 |
| Rv3606c | folK | 2.16 | Rv3581c | ispF | 2.08 |
| Rv0754 | PE_PGRS 11 | 2.16 | Rv3513c | fadD18 | 2.08 |
| Rv2733c | Rv2733c | 2.15 | Rv2628 | Rv2628 | 2.07 |
| Rv1548c | PPE21 | 2.15 | Rv0575c | Rv0575c | 2.07 |
| Rv0347 | Rv0347 | 2.15 | Rv0588 | yrbE2B | 2.07 |
| Rv2135c | Rv2135c | 2.15 | Rv1065 | Rv1065 | 2.07 |
| Rv3504 | fadE26 | 2.15 | Rv1376 | Rv1376 | 2.07 |
| Rv1982A | Rv1982A | 2.14 | Rv2146c | Rv2146c | 2.06 |
| Rv1787 | PPE25 | 2.14 | Rv0064 | Rv0065 | 2.06 |
| Rv1473A | Rv1473A | 2.14 | Rv0897c | Rv0897c | 2.06 |
| Rv3900c | Rv3900c | 2.14 | Rv3526 | Rv3526 | 2.05 |
| Rv0316 | Rv0316 | 2.14 | Rv0456A | mazF1 | 2.05 |
| Rv0511 | hemD | 2.14 | Rv1578c | Rv1578c | 2.05 |
| Rv0663 | vapB8 | 2.14 | Rv2964 | purU | 2.05 |
| Rv2921c | ftsY | 2.14 | Rv1623c | cydA | 2.05 |
| Rv3516 | echA19 | 2.14 | Rv1333 | Rv1333 | 2.05 |
| Rv0114 | gmhB | 2.14 | Rv1213 | glgC | 2.05 |
| Rv2136c | Rv2136c | 2.14 | Rv0711 | atsA | 2.05 |
| Rv3571 | hmp | 2.14 | Rv0452 | Rv0452 | 2.05 |
| Rv0277A | Rv0277A | 2.13 | Rv1102c | Rv1102c | 2.05 |
| Rv2287 | yjcE | 2.13 | Rv0992c | Rv0992c | 2.04 |
| Rv1994c | Rv1994c | 2.12 | Rv2322c | rocD1 | 2.04 |
| Rv0271c | fadE6 | 2.12 | Rv2154c | ftsW | 2.04 |
| Rv3522 | ltp4 | 2.04 | | | |
| Rv1557 | mmpL6 | 2.03 | | | |
| Rv1044 | Rv1044 | 2.03 | | | |
| Rv2660c | Rv2660c | 2.03 | | | |
| Rv1841c | Rv1841c | 2.03 | | | |
| Rv2131c | cysQ | 2.03 | | | |
| Rv0687 | Rv0687 | 2.03 | | | |
| Rv0099 | fadD10 | 2.03 | | | |
| Rv3189 | Rv3189 | 2.03 | | | |
| Rv1958c | Rv1958c | 2.02 | | | |
| Rv1634 | Rv1634 | 2.02 | | | |
| Rv2688c | Rv2688c | 2.02 | | | |
| Rv0303 | Rv0303 | 2.02 | | | |
| Rv3075c | Rv3075c | 2.02 | | | |
| Rv1673c | Rv1673c | 2.02 | | | |
| Rv3065 | mmr | 2.02 | | | |
| Rv3340 | metC | 2.02 | | | |
| Rv1929c | Rv1929c | 2.01 | | | |
| Rv0115 | hddA | 2.01 | | | |
| Rv2503c | scoB | 2.01 | | | |
| Rv0476 | Rv0476 | 2.01 | | | |
| Rv3433c | Rv3433c | 2.01 | | | |
| Rv0875c | Rv0875c | 2.01 | | | |
| Rv1990A | Rv1990A | 2.01 | | | |
| Rv3112 | moaD1 | 2.00 | | | |
| Rv2465c | rpiB | 2.00 | | | |

### Example 4

### Determination of the Protective Efficacy of lsr2 Deletion Mutant of BCG

Immunocompetent BALB/c mice (5 per group, purchased from Charles River Laboratories International, Inc.) were immunized subcutaneously with 5×10⁵ CFU of BCG-Japan, BCG-Japan lsr2 deletion mutant obtained in example 1 and the negative control PBS for 8 weeks. Mice were then challenged by aerosol infection using the Glass-Col Inhalation Exposure System (Glas-Col, LLC) with 300 CFU of M. tb H37Rv. At 5 weeks post infection, 5 mice per group were sacrificed and the lungs were harvested. Harvested lungs were homogenized in 2 mL PBS-0.05% Tween80 using the OMNI TH homogenizer. Lung homogenates were serially diluted, plated in triplicate on 7H11 agar plates and incubated at 37° C. for 4 weeks, and then bacterial colony forming unit (CFU) were counted. The result showed that the lsr2 deletion mutant of BCG (BCG-Japan/Alsr2) exhibits significant better protection than both PBS and its parental strain BCG-Japan (see FIG. 1). *, p<0.05; **, p<0.01.

### REFERENCE LIST

1. Colditz G A, et al. (1995) The efficacy of bacillus Calmette-Guerin vaccination of newborns and infants in the prevention of tuberculosis: meta-analyses of the published literature. Pediatrics 96(1 Pt 1):29-35.
2. Trunz B B, Fine P, & Dye C (2006) Effect of BCG vaccination on childhood tuberculous meningitis and miliary tuberculosis worldwide: a meta-analysis and assessment of cost-effectiveness. Lancet 367(9517):1173-1180.
3. Brewer T F (2000) Preventing tuberculosis with bacillus Calmette-Guerin vaccine: a meta-analysis of the literature. Clin Infect Dis 31 Suppl 3:S64-67.
4. Behr M A (2002) BCG--different strains, different vaccines? Lancet Infect Dis 2(2):86-92.
5. Brandt L, et al. (2002) Failure of the Mycobacterium bovis BCG vaccine: some species of environmental mycobacteria block multiplication of BCG and induction of protective immunity to tuberculosis. Infect Immun 70(2):672-678.
6. Comstock G W (1994) Field trials of tuberculosis vaccines: how could we have done them better? Control Clin Trials 15(4):247-276.
7. Demangel C, Garnier T, Rosenkrands I, & Cole ST (2005) Differential effects of prior exposure to environmental mycobacteria on vaccination with Mycobacterium bovis BCG or a recombinant BCG strain expressing RD1 antigens. Infect Immun 73(4):2190-2196.
8. Fine P E (1995) Variation in protection by BCG: implications of and for heterologous immunity. Lancet 346(8986):1339-1345.
9. Tsenova L, et al. (2007) BCG vaccination confers poor protection against M. tuberculosis HN878-induced central nervous system disease. Vaccine 25(28):5126-5132.
10. Comstock G W, Woolpert S F, & Livesay V T (1976) Tuberculosis studies in Muscogee County, Georgia. Twenty-year evaluation of a community trial of BCG vaccination. Public Health Rep 91(3):276-280.
11. Hart P D & Sutherland I (1977) BCG and vole bacillus vaccines in the prevention of tuberculosis in adolescence and early adult life. Br Med J 2(6082):293-295.
12. Skeiky Y A & Sadoff J C (2006) Advances in tuberculosis vaccine strategies. Nat Rev Microbiol 4(6):469-476.
13. Kaufmann S H (2011) Fact and fiction in tuberculosis vaccine research: 10 years later. Lancet Infect Dis 11(8):633-640.
14. Liu J, Tran V, Leung A S, Alexander D C, & Zhu B (2009) BCG vaccines: their mechanisms of attenuation and impact on safety and protective efficacy. Hum Vaccin 5(2):70-78.
15. Behr M A & Small P M (1997) Has BCG attenuated to impotence?Nature 389(6647):133-134.
16. Gordon B R, Imperial R, Wang L, Navarre W W, & Liu J (2008) Lsr2 of Mycobacterium represents a novel class of H-NS-like proteins. J Bacteriol 190(21):7052-7059.
17. Chen J M, et al. (2006) Roles of Lsr2 in colony morphology and biofilm formation of Mycobacterium smegmatis. J Bacteriol 188(2):633-641.
18. Colangeli R, et al. (2007) Transcriptional regulation of multi-drug tolerance and antibiotic-induced responses by the histone-like protein Lsr2 in M. tuberculosis. PLoS Pathog 3(6):e87.
19. Chen J M, et al. (2008) Lsr2 of Mycobacterium tuberculosis is a DNA-bridging protein. Nucleic Acids Res 36(7):2123-2135.
20. Gordon B R, et al. (2010) Lsr2 is a nucleoid-associated protein that targets AT-rich sequences and virulence genes in Mycobacterium tuberculosis. Proc Natl Acad Sci USA 107(11):5154-5159.
21. Gordon B R, et al. (2011) Structural basis for recognition of AT-rich DNA by unrelated xenogeneic silencing proteins. Proc Natl Acad Sci USA 108(26):10690-10695.
22. Brosch R, et al. (2007) Genome plasticity of BCG and impact on vaccine efficacy. Proc Natl Acad Sci USA 104(13):5596-5601.
23. Cole S T, et al. (1998) Deciphering the biology of Mycobacterium tuberculosis from the complete genome sequence. Nature 393(6685):537-544.
24. Garnier T, et al. (2003) The complete genome sequence of Mycobacterium bovis. Proc Natl Acad Sci USA 100(13):7877-7882.
25. Park K T, et al. (2008) Demonstration of allelic exchange in the slow-growing bacterium Mycobacterium avium subsp. paratuberculosis, and generation of mutants with deletions at the pknG, relA, and lsr2 loci. Appl Environ Microbio 74(6):1687-1695.
26. Bardarov S, et al. (2002) Specialized transduction: an efficient method for generating marked and unmarked targeted gene disruptions in Mycobacterium tuberculosis, M. bovis BCG and M. smegmatis. Microbiology 148(Pt 10):3007-3017.
27. Fleischmann R D, et al. (2002) Whole-genome comparison of Mycobacterium tuberculosis clinical and laboratory strains. J Bacteriol 184(19):5479-5490.
28. Lamm D L (2000) Efficacy and safety of bacille Calmette-Guerin immunotherapy in superficial bladder cancer. Clin Infect Dis 31 Suppl 3:S86-90.
29. Prescott S, Jackson A M, Hawkyard S J, Alexandroff A B, & James K (2000) Mechanisms of action of intravesical bacille Calmette-Guerin: local immune mechanisms. Clin Infect Dis 31 Suppl 3:S91-93.
30. Behr M A, et al. (1999) Comparative genomics of BCG vaccines by whole-genome DNA microarray. Science 284(5419):1520-1523.
31. Glover R T, Kriakov J, Garforth S J, Baughn A D, & Jacobs W R, J r. (2007) The two-component regulatory system senX3-regX3 regulates phosphate-dependent gene expression in Mycobacterium smegmatis. J Bacteriol 189(15):5495-5503.

1. A live modified Mycobacterium bovis-BCG strain, in which lsr2 gene is inactivated or the expression of lsr2 is reduced.

2. (canceled)

3. The live modified Mycobacterium bovis-BCG strain of claim 1, wherein the lsr2 gene is inactivated by deleting the lsr2 gene from the genome.

4. The live modified Mycobacterium bovis-BCG strain of claim 1, wherein the expression of lsr2 is reduced by overexpression of a dominant-negative Lsr2 mutant, expression of antisense Lsr2 transcript, and introducing mutations in the promoter regions of lsr2.

5. The live modified Mycobacterium bovis-BCG strain of claim 1, wherein the Mycobacterium bovis-BCG strain is selected from the group consisting of Mycobacterium bovis-BCG-Russia, Mycobacterium bovis-BCG-Moreau, Mycobacterium bovis-BCG-Japan, Mycobacterium bovis-BCG-Sweden, Mycobacterium bovis-BCG-Birkhaug, Mycobacterium bovis-BCG-Prague, Mycobacterium bovis-BCG-Glaxo, Mycobacterium bovis-BCG-Denmark, Mycobacterium bovis-BCG-Tice, Mycobacterium bovis-BCG-Frappier, Mycobacterium bovis-BCG-Connaught, Mycobacterium bovis-BCG-Phipps, Mycobacterium bovis-BCG-Pasteur and Mycobacterium bovis-BCG-China.

6. A pharmaceutical composition for the treatment or prophylaxis of a mammal against challenge by mycobacteria or against cancer comprising the live modified Mycobacterium bovis-BCG strain of claim 1.

7. The pharmaceutical composition of claim 6, wherein the composition is a vaccine.

8. The pharmaceutical composition of claim 6 wherein the mycobacterium is Mycobacterium tuberculosis or Mycobacterium bovis.

9. The pharmaceutical composition of claim 6, further comprising a pharmaceutically acceptable carrier or an adjuvant or immunogenic materials from one or more other pathogens.

10. A method for the treatment or prophylaxis of a mammal against challenge by Mycobacterium tuberculosis or Mycobacterium bovis, comprising administering to the mammal the live modified Mycobacterium bovis-BCG strain of claim 1 or a pharmaceutical composition containing the live modified Mycobacterium bovis-BCG strain.

11. A method for the treatment or prophylaxis of a mammal against cancer, comprising administering to the mammal the live modified Mycobacterium bovis-BCG strain of claim 1 or a pharmaceutical composition containing the live modified Mycobacterium bovis-BCG strain.

12. The method of claim 10, wherein the mammal is a cow or a human.

13. The method of claim 11, wherein the cancer is bladder cancer.

14. (canceled)

15. The method of claim 11, wherein the mammal is a cow or a human.
